(19) United States
(12) Patent Application Publication
(10) Pub. No.: US 2017/0369384 A1
(43) Pub. Date: Dec. 28, 2017

(54) DRYING APPARATUS FOR DRYING A SUBSTANCE, POULTRY MANAGEMENT SYSTEM AS WELL AS METHOD OF DRYING A SUBSTANCE

(71) Applicant: BIG DUTCHMAN INTERNATIONAL GMBH, Vechta (DE)

(72) Inventor: Ludger THEMANN, Vechta (DE)

(73) Assignee: BIG DUTCHMAN INTERNATIONAL GMBH, Vechta (DE)

(21) Appl. No.: 15/621,156

(22) Filed: Jun. 13, 2017

(30) Foreign Application Priority Data
Jun. 27, 2016 (DE) 20 2016 103370.5

(51) Int. Cl. C05F 3/06; F26B 17/00; F24F 11/00; C02F 11/12; F26B 3/04; G01N 33/00; F26B 5/04; C05F 3/00

(52) U.S. Cl. CPC C05F 3/06; G01N 33/0054; F26B 17/008; F26B 5/041; F24F 11/001; C02F 11/12; F26B 3/04; C05F 3/00; F26B 17/00

(57) ABSTRACT

The present invention relates to a drying apparatus for drying a substance, in particular for drying poultry manure, comprising a drying unit with a conveyor, a dry substance sensor and an ammonia sensor.

### CROSS-REFERENCE TO FOREIGN PRIORITY APPLICATION

The present application claims the benefit under 35 U.S.C. §119(b) of German Application No. DE 20 2016 103370.5 filed Jun. 27, 2016, titled “Drying Apparatus for Drying a Substance, Poultry Management System as well as Method of Drying a Substance.”

### FIELD OF THE INVENTION

The present disclosure relates to a drying apparatus for drying a substance, a control system for a drying apparatus for drying a substance, a drying system, a poultry management system, and a method of drying a substance.

### BACKGROUND OF THE INVENTION

Farm animals, for example, poultry, such as laying hens, broilers, turkey hens, or ducks, are usually kept in farming businesses, wherein housing systems, in particular, are used. These farming businesses produce, in particular, in the housing systems of the farming businesses, animal products. In modern, highly modernized housing systems, the environment of the animals is optimized in order to, for example, allow for species-appropriate animal management and, at the same time, efficient production. As used herein, the term “environment” means, for example, the air in the house and/or the conditions on the house floor. In the following, the term “animal products” shall mean in particular the by-products of the actual production, such as manure, and, in particular poultry manure and/or feathers, which are produced, for example, in the context of rearing, egg production or meat production.

One component of modern housing systems is conveyors for transporting animal products. The conveyors may, for example, be configured as belt conveyors or as slat conveyors. A belt conveyor will usually comprise a conveyor belt, in most cases a continuous conveyor belt, and a belt drive which drives the conveyor belt into one or several transport directions. Instead of a belt conveyor, a slat conveyor may be provided, which essentially differs from a belt conveyor in that a number of connected slats is used instead of the conveyor belt. Due to the similarity between belt and slat conveyors with regard to the presently relevant topic, in the following, the terms “belt conveyor” and “conveyor belts” shall also comprise slat conveyors and conveyor slats, respectively. Conveyors serve, in particular, the purpose of transporting animal products from various animal management systems away for disposal or further processing in a fast and reliable manner. In the case of conveyors for transporting substances to be dried, such as poultry manure, it may be additionally required that the substance be aired in order to dry it or to dry it in separate drying apparatuses, in which the previously described conveyors can be used as well.

The drying of the animal products, of poultry manure, in particular, is prerequisite for making it possible to store said animal products well and to further market them. Drying tunnels in which air, for example, the warm exhaust air from a house, is guided via fans in a pressure corridor, are preferably used for drying. Above and next to the pressure corridor, preferably several levels of perforated conveyor belts, also referred to as drying belts, are arranged in such a manner that the exhaust air can enter through openings into the drying tunnel and flow through the perforation so that the moisture can be extracted from the animal product and the animal product can be dried. The drying may take place in a closed space if the air emitted by the drying tunnel is to be filtered, which allows for making the drying process mostly odor-neutral. The precondition of optimum drying is that the belts are loaded with the animal product to be dried as evenly as possible.

A consistent drying of the animal product to be dried, the poultry manure, in particular, has positive effects on the storability and marketing of the substance. Especially, consequential costs can thus be reduced. A consistently dried substance, poultry manure in particular, which preferably has a high dry substance percentage is, in particular, considered to be of higher quality. The drying of the substance is impacted by different parameters. In addition to the height of the substance on a conveyor belt, for example, the initial moisture content, the temperature and humidity of the drying air and the duration of the drying process are relevant. Often, these parameters are, to a large extent, uncontrollable or are only controllable with limitations so that the drying process will usually not deliver consistent results, and, therefore, especially the moisture content of the animal products at the end of the drying process will vary significantly. While the existing apparatuses and methods of operating such apparatuses offer various advantages, further improvements are desirable.

Therefore, it is the object of the present invention to propose a solution which reduces or eliminates one or several of the above mentioned disadvantages. It is, in particular, an object of the present invention to propose a solution which provides improved manure removal and/or an improved climate of a house. In addition, it is the object of the present invention to propose a solution which allows for species appropriate and/or efficient animal management.

### SUMMARY OF THE INVENTION

Pursuant to a first aspect of the present invention, the above-mentioned object is achieved by means of a drying apparatus for drying a substance, such as animal products, and in particular, for drying poultry manure, comprising a drying unit with a conveyor, a dry substance sensor, and an ammonia sensor.

Preferably, the drying unit comprises a substance inlet and a substance outlet. The substance inlet is, in particular, arranged and designed to transport and/or guide the incoming substance to be dried, in particular, poultry manure, which has preferably been transported out of a house to a drying apparatus, into the drying unit. The substance outlet is preferably arranged and designed to transport and/or guide the dried substance, in particular, dried poultry manure, out of the drying unit. An advantageous embodiment of the drying unit further requires that said drying unit comprises two or more substance inlets and/or two or more substance outlets. Accordingly, the drying unit comprises at least one substance inlet and at least one substance outlet. It is preferable that a drying area is arranged between the at least one substance inlet and the at least one substance outlet.

The substance to be dried is preferably transported from the one substance inlet to a conveyor which transports the substance to be dried through the drying area on a preferably predetermined movement path through the drying area to the at least one substance outlet. Preferably, the conveyor comprises at least one conveyor belt, which is preferably designed as a continuous belt. Furthermore, it is preferable that the conveyor comprises two, three, or more conveyor belts, which are preferably arranged vertically on top of each other, wherein said conveyor belts are preferably partially offset from each other in the horizontal direction and/or the transport direction so that the substance to be dried can reach a lower conveyor belt from an upper conveyor belt. Preferably, the transport direction of the upper conveyor belt is opposite to the transport direction of the lower conveyor belt. Such an arrangement allows for the substance to be dried to move preferably several times through a drying area, in which the conveyor belts are preferably arranged on top of each other. The entire distance that the substance covers when moving through the drying unit is referred to as the “drying distance.”

Pursuant to the invention, the drying apparatus comprises a dry substance sensor. The dry substance sensor can determine the dry substance portion of a substance. This portion is also referred to as the “dry substance content” (DS content). Especially poultry manure usually contains non-dry substances, i.e., substances which are liquid, in addition to dry substances, i.e., solid substances. The liquid portion usually consists mostly of water. Dry substance sensors may be designed tactile or non-tactile. Furthermore, the dry substance sensors may also be used as hybrid variations with tactile and non-tactile functions. The dry substance sensor is preferably arranged in an area adjacent to the conveyor. Furthermore, it is preferable that the dry substance sensor is arranged in such a way that it can make contact with the substance to be dried.

It is, in particular, preferable that the dry substance sensor is arranged in an area adjacent to the movement path of the substance between the substance inlet and the substance outlet. In a particularly preferred embodiment, the drying apparatus comprises two or more dry substance sensors.

Furthermore, the drying apparatus comprises an ammonia sensor. The ammonia sensor measures especially the ammonia content of the air flowing through, in particular of the air flowing through the drying unit. This allows for a determination of the ammonia evaporation from the substance to be dried, the poultry manure, in particular, for example, during the drying process. In addition, the ammonia sensor makes it possible to prove how high the actual ammonia emission of the house is.

Ammonia is a colorless, water soluble, and, in particular, poisonous gas with a strong, sharp smell of which only a small percentage should be contained in the house air. In order to ensure a species-appropriate management of the animals and, furthermore, the wellbeing of the animals, a reduction of the ammonia content in the house air is desirable. The ammonia sensor is preferably arranged inside the drying unit. Furthermore, it is preferable that the drying apparatus comprises two or more ammonia sensors arranged at several locations in the drying unit. Such an arrangement of several ammonia sensors has the advantage that an average value of the ammonia load can be determined via the drying unit. Furthermore, it is preferable that the several ammonia sensors are arranged along the movement path of the substance to be dried inside the drying unit.

It is, in particular, preferable that in relation to the movement path, one dry substance sensor and/or one ammonia sensor are arranged at a shorter distance from the substance outlet than from the substance inlet. It is, in particular, preferred that one dry substance sensor and/or one ammonia sensor are arranged in an area of the movement path adjacent to the substance outlet, wherein the distance to the substance outlet in relation to the movement path is smaller than 50%, smaller than 40%, smaller than 30%, smaller than 20%, smaller than 10%, or smaller than 5% of the drying distance.

It is furthermore preferable that, in relation to the movement path, one dry substance sensor and/or one ammonia sensor are arranged at a shorter distance from the substance inlet than from the substance outlet. It is, in particular, preferred that one dry substance sensor and/or one ammonia sensor are arranged in an area of the movement path adjacent to the substance inlet, wherein the distance to the substance inlet in relation to the movement path is smaller than 50%, smaller than 40%, smaller than 30%, smaller than 20%, smaller than 10% or smaller than 5% of the drying distance.

Furthermore, several dry substance sensors and/or several ammonia sensors may preferably be arranged at an equal distance in relation to the movement path.

A preferred embodiment of the drying apparatus requires that the dry substance sensor is designed as a NIR sensor and/or a microwave sensor. NIR is short for near-infrared spectroscopy, which is a physical analysis technique on the basis of the spectroscopy in the range of short-wave infrared light. It essentially corresponds to the infrared spectroscopy used in the medium and far infrared range (MIR and FIR).

The dry substance sensor has preferably a measurement range of 0-85% residue moisture and/or a measurement accuracy of less than one percent absolute, in particular, of approx. 0.5% absolute. Furthermore, the dry substance sensor is preferably suitable for a temperature of the product to be dried from 0° C. to +80° C.

Pursuant to another preferred embodiment of the drying apparatus, it is required that the dry substance sensor is arranged in a height-adjustable manner. This means, in particular, that the dry substance sensor is arranged in such a way that it can be moved vertically and/or in a direction orthogonal to the preferably planar extension of the conveyor, in particular of a conveyor belt. A dry substance sensor adjustable in height especially has the advantage that the dry substance content can always be determined in, preferably, direct proximity to the surface of the substance to be dried. Preferably, the dry substance sensor is arranged above the substance to be dried.

Compared to an arrangement of a dry substance sensor below the conveyor and/or an arrangement at a clear distance from the substance to be dried (for example, high above the substance), the provided height-adjustable arrangement has the advantage that the position of the dry substance sensor can be adjusted to a varying substance height so that relevant and/or reliable measurement values can be generated by means of the dry substance sensor. It is, in particular, preferred that the height adjustability is executed in dependence on the height of the substance to be dried on the conveyor.

Pursuant to another preferred embodiment of the drying apparatus, it is required that the dry substance sensor is arranged and configured in such a way that, even in case of different substance heights on the conveyor belt, the dry substance sensor is essentially located directly on top of the substance. “On top of the substance” means, in particular, that the dry substance sensor is located on top of or in, preferably, direct proximity to the surface of the substance to be dried.

Pursuant to another preferred embodiment of the drying apparatus, it is required that the dry substance sensor is arranged at a suspension attachment, which is preferably arranged above the conveyor. By means of the dry substance sensor arranged at a suspension attachment, it can be ensured or improved that the dry substance sensor is arranged at a predefined and/or varying height above the conveyor. By means of a suspension attachment, it can, in particular, be ensured that the dry substance sensor is essentially always arranged directly on top of the substance and/or on top of the surface of the substance even in case of varying substance heights on the conveyor.

Pursuant to a particularly preferred embodiment of the drying apparatus, it is required that the suspension attachment comprises a slide and/or that the suspension attachment comprises a guiding element, which is preferably installed in a swivelable manner at its first and/or its second end. The dry substance sensor is preferably arranged at the slide. The guiding element is preferably essentially rod-shaped. Preferably, one end of the guiding element is arranged at a static component of the drying apparatus and the other end at the slide.

The provision of a slide has the advantage that, even in the usual case of a high density of the substance to be dried in the house installations, the slide, preferably equipped with a dry substance sensor, is essentially arranged on top of the surface of the substance. The slide has preferably a planar extension with a bottom side and a top side. Orthogonally to the planar extension, the slide preferably has on at least one of its edges one, two, or several side walls, which preferably have a planar extension orthogonal to the top and/or bottom side. It is particularly preferred that the slide has a side wall on the edge, the planar extension of which is slanted, for example, by between 5° and 90°, in particular between 30° and 60°, for example, by 45°, towards the transport direction.

Preferably, the side walls are slanted outwards from the center of the slide. Furthermore, it is ensured or improved through these slanted side walls that the slide is arranged on top of the surface of the substance to be dried and also remains there when the conveyor unit with the substance to be dried is moved in the transport direction in relation to the slide. The bottom surface of the slide preferably faces the substance to be dried. The top surface of the slide is preferably facing away from the substance to be dried.

Preferably, the dry substance sensor is arranged on the top surface of the slide. In addition, the slide may have a gap in the place where the dry substance sensor is arranged at the slide so that the dry substance sensor can have direct contact with the substance to be dried on the bottom side of the slide.

Such a suspension attachment with a guiding element and a slide allows for the cost-efficient and, in particular, safe arrangement of a dry substance sensor on top of the surface of the substance to be dried, without necessarily requiring a complex control device, comprising, for example, a height sensor and/or an automatic adjustment of the height of the dry substance sensor. The rotation axes of the guiding element of the suspension attachment and/or of the slide are preferably arranged essentially parallel and/or essentially orthogonal to the conveyor.

Pursuant to another particularly preferred embodiment of the drying apparatus, it is required that the drying apparatus comprises one or several sensors which are configured to determine one or several additional substance parameters, such as the nitrogen, nitrogen oxide, phosphorus, or protein content of the substance. Preferably, the dry substance sensor is arranged and configured to determine one or several substance parameters, such as the nitrogen, nitrogen oxide, phosphorus, or protein content of the substance.

Pursuant to another particularly preferred embodiment of the drying apparatus, it is required that the drying apparatus comprises a volume flow sensor which is configured and arranged to measure a fluid flowing through the drying apparatus, for example air. In a preferred combination with the ammonia sensor, a so-called ammonia load can be determined, which can be summarized and evaluated in a house balance. The ammonia load is, for example, the ammonia amount transported out of the house per time unit. Furthermore, it is preferable that the drying apparatus comprises at least one ventilation installation in order to allow for a circulation of air within the drying apparatus or in exchange with the environment.

Pursuant to another particularly preferred embodiment of the drying apparatus, it is required that any of the dry substance sensor, ammonia sensor, and/or volume flow sensor is arranged and configured to transfer sensor data to a control unit. Preferably, the control unit is configured and arranged to control the drying apparatus or a drying system in such a way that the desired conditions, for example with regard to the ammonia content or the dry substance content in the substance to be dried, are influenced.

Pursuant to another particularly preferred embodiment of the drying apparatus, it is required that the control parameter comprises one or several of the following parameters: time required for drying the substance, maximum and/or minimum height of the substance on the drying unit and/or on a transport system, amount and/or volume of a recirculated or vented fluid in the drying apparatus, speed of the conveyor and/or the manure transport units, a start and/or stop signal for the conveyor and/or manure transport unit.

Pursuant to another particularly preferred embodiment of the drying apparatus, it is required that the control unit is configured to determine a balance and/or total amount of the substances emitted in a predefined period of time, in particular ammonia and/or nitrogen and/or phosphorus and/or recirculated fluid and/or vented fluid, based on the sensor data. Thus, a balance can be created for the house, which can also be referred to as a total balance or house balance and which shows, in particular, which substances, in particular undesired substances or harmful substances, respectively, are and/or were emitted. Preferably, the control unit is arranged and configured to influence the different parameters of the drying apparatus on the basis of the house balance or the above-mentioned sensor data, respectively, so that a house balance is within the limits of the desired values.

Furthermore, it is preferable that the control unit is configured to receive and/or evaluate at least one or several of the following parameters: a load of the conveyor, a load of conveyor arranged in a house, or environmental parameters in the drying apparatus and/or in a farm animal house, for example the ammonia content of the air and/or the temperature and/or air humidity. Furthermore, it is preferred that the control unit is configured to generate at least one control signal with which the drying apparatus or manure transport units in a farm animal house can be controlled.

In addition, it is preferred that the control unit is configured as self-learning and/or configured to generate prognoses from previous data. Thus, for example, manure removal intervals for the future can be derived. Furthermore, it is preferred that the control unit is configured to execute a fully automatic manure removal for a farm animal house.

Pursuant to another preferred embodiment, the drying apparatus comprises a load sensor.

A load sensor serves preferably the purpose of determining the load of the substance to be dried on the conveyor. Preferably, the speed and/or the speed at which the substance to be dried is input into the substance inlet, the drying duration, and/or additional parameters relevant to the manure removal and/or the drying process can be controlled in dependence on the load determined by the load sensor.

Pursuant to another aspect of the invention, the above mentioned object is achieved through a control system for a drying apparatus for drying a substance, in particular for drying poultry manure, preferably for a drying apparatus pursuant to at least one of the embodiments described above, comprising a control unit which is arranged and configured to receive sensor data from one or several sensors, in particular, from a dry substance sensor, an ammonia sensor, a volume flow sensor, and/or a load sensor of a manure transport installation, and to derive from the sensor data one or several control parameters for the operation of the drying apparatus and/or of a manure transport installation, and to transmit the control parameter or control parameters to a drying apparatus and/or one or several manure transport installations. The control parameters comprise preferably one or several of the following parameters: time required for drying the substance, maximum and/or minimum height of the substance in and/or on the drying unit and/or a transport system, amount and/or volume of a recirculated and/or vented fluid in the drying apparatus, speed of the conveyor and/or the manure transport units, start and/or stop signal for the conveyor and/or manure transport units. It is, in particular, possible to control the drying apparatus and/or one or several manure transport installations on the basis of these control units in such a way that the ammonia content in the house air and/or the dry substance content of the substance to be dried reaches, in particular at the substance outlet, one or several desired values.

In a preferred embodiment of the control system, it is required that the control unit is arranged and configured to take into account one or several peripheral conditions for the determination of the control parameter or control parameters, such as a down time of the drying apparatus, or of one or several manure transport installations during the operation of egg collection and/or egg transport installations, and/or one or several user settings. In another advantageous embodiment, the control system has a user interface for entering user settings. In the present case, user settings may be, for example, the dry substance percentage and/or the ammonia percentage or, respectively, the ammonia amount in the exhaust air of the house and/or the drying area and/or the drying apparatus.

Pursuant to another aspect of the invention, the object mentioned above is achieved through a drying system comprising a drying apparatus pursuant to at least one of the above described embodiments and a control system pursuant to at least one of the embodiments described above. In a particularly preferred embodiment of the drying system, it is required that the drying apparatus is arranged outside a poultry house. The arrangement of the drying apparatus outside the poultry house makes it possible to remove animal products, such as ammonia and/or poultry droppings, quickly from the poultry house and to not have the animals in the poultry house exposed to the contamination with the animal products anymore or to only have them exposed to a reduced extent.

Pursuant to another aspect of the present invention, the object mentioned above is achieved through a poultry management system comprising a drying apparatus pursuant to at least one of the embodiments described above, a control system pursuant to at least one of the embodiments described above, a drying system pursuant to at least one of the embodiments described above, and/or one or several manure transport installations. In a particularly preferred embodiment of the poultry management system, it is required that the drying apparatus is arranged outside a poultry house.

Pursuant to another aspect of the invention, the object mentioned above is achieved through a method of drying a substance comprising the steps of providing a drying unit with a conveyor, and determining the dry substance content and/or ammonia emission in the conveyor.

The method pursuant to the invention and its possible realizations comprise features or respectively method steps which make it, in particular, suitable for being used for a drying apparatus pursuant to the invention, a control system, a drying system and/or a poultry management system and their respective embodiments. Regarding the further advantages, possible versions and realization details of these additional aspects and their realizations, reference is also made to the previous description of the respective features and embodiments of the drying apparatus.

As regards the advantages, embodiment variants, and embodiment details of the method according to the present disclosure and its possible further developments, reference is made to the description provided herein of the respective features, as will be understood and appreciated by those skilled in the art upon studying the following specification, claims, and appended drawings.

### BRIEF DESCRIPTION OF THE DRAWINGS

Preferred embodiments of the invention are, by way of example, described by means of the attached figures. The following is shown in

FIG. 1 is a side schematic view of a first exemplary embodiment of a drying apparatus;

FIG. 2 is a front perspective view of an exemplary embodiment of a suspension attachment with a slide;

FIG. 3 is a side schematic view of another exemplary embodiment of a drying apparatus; and

FIG. 4 is a top schematic view an exemplary embodiment of a poultry management system.

### DETAILED DESCRIPTION OF THE PREFERRED EMBODIMENTS

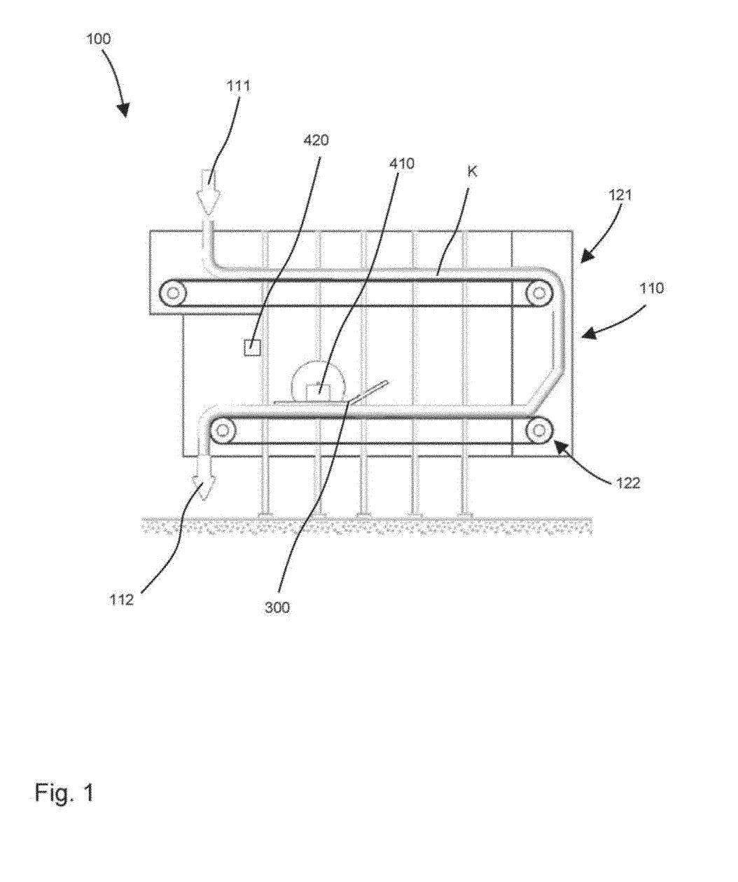

As referenced in the Figures, the same reference numerals may be used herein to refer to the same parameters and components or their similar modifications and alternatives. For purposes of description herein, the terms “upper,” “lower,” “right,” “left,” “rear,” “front,” “vertical,” “horizontal,” and derivatives thereof shall relate to the present disclosure as oriented in FIG. 1. However, it is to be understood that the present disclosure may assume various alternative orientations and step sequences, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise. The drawings referenced herein are schematic and associated views thereof are not necessarily drawn to scale. Identical elements or elements with essentially the same or similar function are referred to with the same reference number in the Figures.

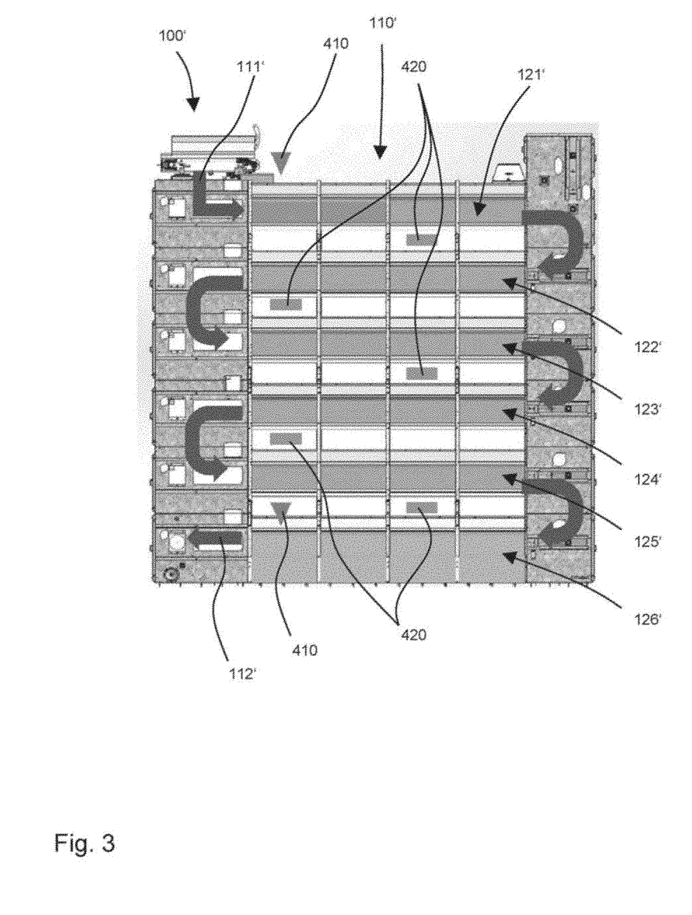

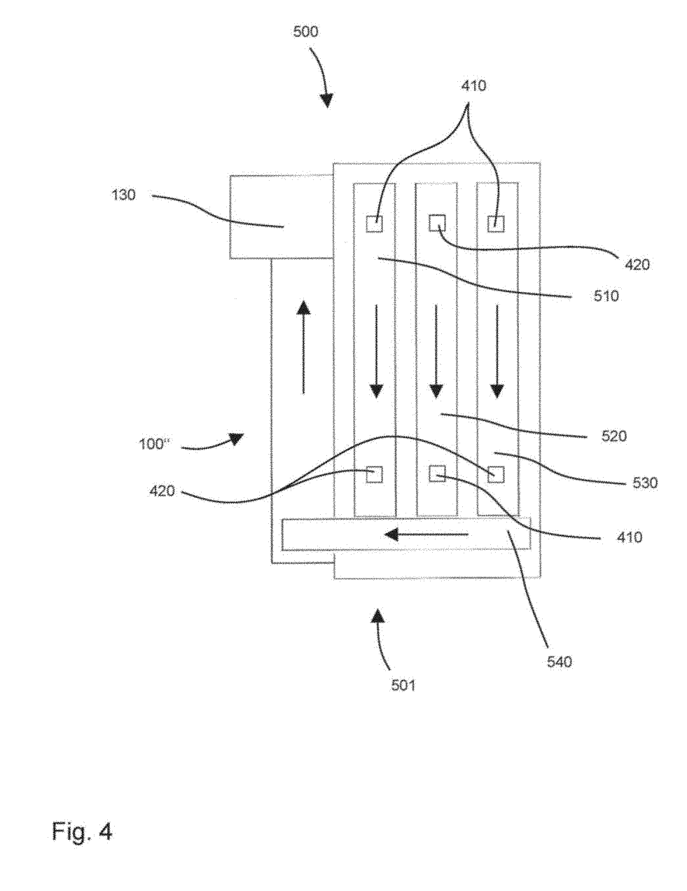

FIG. 4 shows a poultry management system 500 with a poultry house 501 in which several manure transport units 510, 520, 530, 540 are arranged. The transport directions of the manure transport units 510, 520, 530, 540 are marked with arrows. The three manure transport units 510, 520, 530 are arranged parallel to each other and serve the purpose of transporting products away from animal holding areas (not shown) also arranged in rows in a house. At the beginning and the end of the three manure transport units 510, 520, 530, dry substance sensors 410 and ammonia sensors 420 are arranged, respectively. The three manure transport units 510, 520, 530 transport the manure onto another manure transport unit 540, arranged transverse to said three manure transport units 510, 520, 530. This manure transport unit 540 serves the purpose of transporting the manure produced in the entire poultry house 501 to a drying apparatus 100″, which is arranged outside the poultry house. After the manure has left the drying apparatus 100″ essentially in the transport direction marked with an arrow in FIG. 4, the dried manure may be intermediately stored in the intermediate storage installation 130 or may also be directly packaged and/or transported away. The drying apparatus 100″ may, for example, be configured pursuant to the exemplary embodiments of drying apparatuses 100, 100′ shown in FIGS. 1 and 3, respectively.

FIGS. 1 and 3 show exemplary embodiments of drying apparatuses 100, 100′. The drying apparatus 100 shown in FIG. 1 comprises a drying unit 110 with two conveyors 121, 122, which transport the manure K from a substance inlet 111 along a movement path to the substance outlet 112. In this case, the transport directions of the two conveyors 121, 122 are opposite to each other.

The drying apparatus 100′ shown in FIG. 3 comprises a conveyor unit 110′ with six conveyors 121′, 122′, 123′, 124′, 125′, and 126′, which transport the manure from a substance inlet 111′ to a substance outlet 112′. The transport directions of two neighboring conveyors 121′, 122′, 123′, 124′, 125′, and 126′, respectively, are preferably opposite to each other. As illustrated in FIG. 3 by means of the arrows, the movement path of the manure is meander shaped.

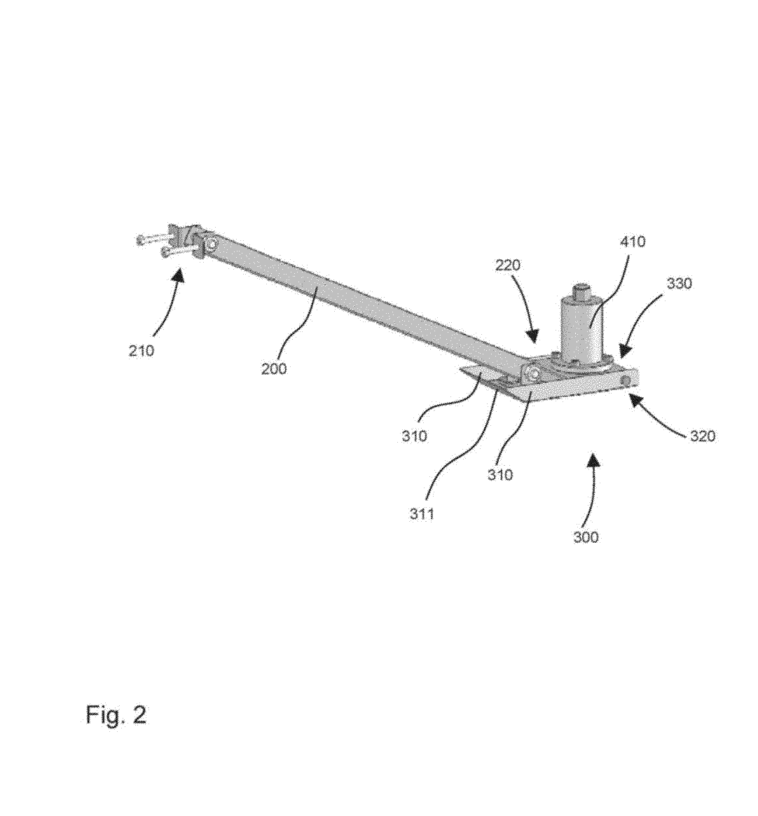

In the conveyor 100′ pursuant to FIG. 3, two dry substance sensors 410 are arranged in the vicinity of the substance inlet 111′ and in the vicinity of the substance outlet 112′. Furthermore, four moisture and temperature sensors 420′ are arranged between the dry substance sensors 410 and are provided along the movement path. The conveyor 100 pursuant to FIG. 1 shows a dry substance sensor 410 and a humidity and temperature sensor 420′. In the two drying apparatuses 100,100′, the dry substance sensors 410 are preferably arranged on a slide 300 (shown in FIGS. 1 and 2).

FIG. 2 shows a slide 300 with its suspension attachment 200 in detail. The suspension attachment 200 comprises at its first end 210 and at its second end 220 attachment elements with which the slide 300 can be attached at the second end 220 in a swivelable manner. At the first end 210, the suspension attachment 200 can be attached in a swivelable manner to the drying apparatus. The rotation axes for the swivel movement of the suspension attachment 200 at its first end 210 and its second end 220 are preferably essentially horizontal and, furthermore, essentially orthogonal to the transport direction.

On a top surface 330 of the slide 300, a dry substance sensor 410 is arranged. Preferably, the dry substance sensor 410 extends through a gap in the slide 330 so that a sensor element of the dry substance sensor 410 can preferably be arranged on the bottom surface 320 of the slide 300 in direct proximity to or on a surface of the manure to be dried. The side walls 310 of the slide 300 arranged parallel to the transport direction are slanted orthogonally to the bottom surface 320. Another side wall 311 is preferably slanted at an angle of approx. 45° to the transport direction.

The suspension attachment 200 allows for a height-adjustable attachment of the dry substance sensor 410 so that the dry substance sensor 410 can simply and without requiring extensive control effort follow the load of the conveyor with manure as the substance to be dried. The slide 300, in particular, with its front side wall 311 slanted towards the transport direction, serves the purpose of sliding along on the manure and also of ensuring that the dry substance sensor 410 is on top of or in direct proximity to the surface of the manure.

The determination of the dry substance content by means of the dry substance sensor 410 and the simultaneous determination of the ammonia evaporation by means of an ammonia sensor 420 allows first of all for an improvement of the drying process and/or the quality of the dried product obtained at the end of the drying process. Furthermore, the data gained through the sensors may serve for the preparation of a house balance and/or the determination of an overall amount of emissions of the house. Thus, for example manure removal intervals can be controlled in such a way that the emission values in the house will not exceed certain threshold values, which may significantly increase the wellbeing of the animals. At the same time, the drying process can be adjusted to the moisture contained in the product to be dried and/or peripheral parameters, such as the ambient temperatures and/or air humidity, so that an end product of high, consistent quality can be achieved in an efficient drying process and, therefore, also with an efficient use of resources.

It will be understood by one having ordinary skill in the art that construction of the described present disclosure and other components is not limited to any specific material. Other exemplary embodiments of the disclosure disclosed herein may be formed from a wide variety of materials, unless described otherwise herein.

For purposes of this disclosure, the term “operably coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

For purposes of this disclosure, the term “operably connected” (in all of its forms, connect, connecting, connected, etc.) generally means that one component functions with respect to another component, even if there are other components located between the first and second component, and the term “operable” defines a functional relationship between components.

It is also important to note that the construction and arrangement of the elements of the present disclosure as shown in the exemplary embodiments is illustrative only. Although only a few embodiments of the present innovations have been described in detail in this disclosure, those skilled in the art who review this disclosure will readily appreciate that many modifications are possible, e.g., variations in sizes, dimensions, structures, shapes and proportions of the various elements, values of parameters, mounting arrangements, use of materials, colors, orientations, etc. without materially departing from the novel teachings and advantages of the subject matter recited. For example, elements shown as integrally formed may be constructed of multiple parts or elements shown in multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of the wide variety of materials that provide sufficient strength or durability, in any of the wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

It is to be understood that variations and modifications can be made on the aforementioned structure and method without departing from the concepts of the present disclosure, and further it is to be understood that such concepts are intended to be covered by the following claims unless these claims by their language expressly state otherwise.

1-15. (canceled)

16. A drying apparatus for drying a substance, in particular for drying poultry manure, comprising a drying unit with a conveyor, a dry substance sensor, and an ammonia sensor.

17. A drying apparatus pursuant to claim 16, wherein the dry substance sensor is an NIR sensor or a microwave sensor.

18. A drying apparatus pursuant to claim 16, wherein the dry substance sensor is arranged in a height-adjustable manner.

19. A drying apparatus pursuant to claim 16, wherein the dry substance sensor remains substantially located directly on top of the substance regardless of substance height on the conveyor belt.

20. A drying apparatus pursuant to claim 16, wherein the dry substance sensor is arranged at a suspension attachment disposed above the conveyor.

21. A drying apparatus pursuant to claim 20, wherein the suspension attachment comprises a slide and a guiding element installed in a swivelable manner on one or more of a first or a second end thereof.

22. A drying apparatus pursuant to claim 16, wherein the dry substance sensor is arranged and configured to determine one or several additional substance parameters.

23. A drying apparatus pursuant to claim 22, wherein the additional substance parameter is a nitrogen and/or a phosphorus content of the substance.

24. A drying apparatus pursuant to claim 16, further comprising a volume flow sensor adapted to measure a fluid flowing through the drying apparatus.

25. A drying apparatus pursuant to claim 24, wherein the fluid is air.

26. A drying apparatus pursuant to claim 24, wherein one or more of the dry substance sensor, the ammonia sensor, or the volume flow sensor is adapted to transfer sensor data to a control unit.

27. A drying apparatus pursuant to claim 16, further comprising a control unit and wherein a control parameter comprises one or more of the following parameters:
time required for drying the substance;
maximum and/or minimum height of the substance on the drying unit and/or on a transport system;
amount and/or volume of a recirculated and/or vented fluid in the drying apparatus;
speed of the conveyor and/or the manure transport unit(s); and
start and/or stop signal for the conveyor and/or manure transport unit(s).

28. A drying apparatus pursuant to claim 27, wherein the control unit is configured to determine a balance and/or total amount of the substances emitted in a predefined period of time, based on the sensor data.

29. A drying apparatus pursuant to claim 28, wherein the substances may include ammonia, nitrogen, phosphorus, and/or recirculated fluid or vented fluid.

30. A control system for a drying apparatus for drying a substance, in particular for drying poultry manure, preferably for a drying apparatus pursuant to claim 16, the control system comprising a control unit adapted to receive sensor data from one or more sensors, selected from the group consisting of a dry substance sensor, an ammonia sensor, a volume flow sensor, and a load sensor of a manure transport installation to derive from the sensor data one or several control parameters for the operation of a drying apparatus or a manure transport installation, and to transmit the one or more control parameters to a drying apparatus and/or one or more manure transport installations.

31. A drying system comprising a drying apparatus according to claim 16, further comprising a control system comprising a control unit adapted to receive sensor data from one or more sensors, selected from the group consisting of a dry substance sensor, an ammonia sensor, a volume flow sensor, and a load sensor of a manure transport installation to derive from the sensor data one or several control parameters for the operation of a drying apparatus or a manure transport installation, and to transmit the one or more control parameters to a drying apparatus and/or one or more manure transport installations.

32. A poultry management system comprising a drying apparatus pursuant to claim 31 adapted for use on one or more manure transport installations.

33. A method of drying a substance, comprising the steps of providing a drying unit with a conveyor, determining a dry substance content and/or ammonia emission in the conveyor.
